(19) United States
(12) Patent Application Publication
(10) Pub. No.: US 2017/0165195 A1
(43) Pub. Date: Jun. 15, 2017

(54) EDIBLE CRICKET COMPOSITION FOR PREVENTING HAIR LOSS, IMPROVING HAIR FOLLICLE, AND PROMOTING HAIR REGROWTH

(71) Applicant: Sam Goo LEE, Jeonju-si (KR)

(72) Inventor: Sam Goo LEE, Jeonju-si (KR)

(21) Appl. No.: 15/367,979

(22) Filed: Dec. 2, 2016

(30) Foreign Application Priority Data
Dec. 9, 2015 (KR) 10-2015-0174892
Oct. 6, 2016 (KR) 10-2016-0129339

(51) Int. Cl. A61K 8/98; A61Q 7/00

(52) U.S. Cl. CPC A61K 8/987; A61Q 7/00; A61K 2800/92

(57) ABSTRACT

A hair regrowth is developed using edible crickets. The edible crickets are provided in the form of 60-100 g of powders or in the form of 60-100 g of formulations on a daily basis. The edible crickets reduce the concern about side effects and are safe. The intake rate of the edible crickets is higher than those of synthetic medicine of promoting hair regrowth and medicine of preventing hair loss, so that the edible crickets are used for pharmaceuticals, supplements, and cosmetics. The edible crickets contains iron (Fe), zinc (Ze), copper (Cu), and biotin (Biotin, Vitamin B7) higher than nutritional ingredients contained in vitamins sold on the market in content, and a large amount of amino acids constituting hairs, so that an excellent effect is produced on hair regrowth. The promotion of hair regrowth, the prevention of hair loss, and the improvement of hair follicles are easily recognized.

### CROSS-REFERENCE TO RELATED APPLICATIONS

This application claims the benefit of Korean Application No. 10-2015-0174892 filed on Dec. 9, 2015, and Korean Application No. 10-2016-0129339 filed on Oct. 6, 2016, the disclosures of which are incorporated herein in their entirety by reference thereto.

### 1. FIELD OF THE INVENTION

The present invention relates to a composition containing effective ingredients of edible crickets to promote hair regrowth, prevent hair loss, or improve hair follicles. More particularly, the present invention provides a composition capable of minimizing side effects while preventing hair loss, promoting hair regrowth, or improving hair follicles using powders or extracts of edible crickets.

### 2. DESCRIPTION OF THE RELATED ART

Hair on scalp, which protects a part of our body, not only protect a head from various physical impacts including an external stimulus, ultraviolet rays, cold, and heat, but also express the image of an outer appearance. However, unexpected hair loss severely exerts an influence on the function of the hair.

It has been perceived in the past that hair loss is known as being genetically influenced, and baldness is shown on a family line. However, it is known that the hair loss is affected by various causes including a genetic cause in modern times. In other words, it has become known that hair loss is also caused by excessive smoking, stress, insomnia, or hormonal imbalance. However, the exact cause of the hair loss has not yet been clarified, and there is no reliable manner for preventing the hair loss. In this situation, as the stress of modern people due to hair loss is generally increased, the interest in an effective technology of preventing hair loss and promoting hair growth is increased. Currently, a variety of hair loss prevention agents and hair growth agents including shampoo are commercially available.
(Patent document 0001) Korean Unexamined Patent Publication No. 10-2011-0138776.
(Patent document 0002) Korean Patent Registration No. 10-1147288.

Most related arts including the above cited references have features of promoting hair regrowth as a user applies mixed extracts of medical plants to scalp or takes in the mixed extracts. However, the mixed extracts of the medical plants have insufficient effects on the prevention of hair loss or the promotion of the hair regrowth. Especially, when hundreds of thousands of patients subject to hair transplantation and treatment each year directly apply natural extracts, which are developed until now, to their scalp, side effects, such as skin irritation or damage, occur. In addition, the risk of side effects, such as the decrease of a sexual function, the decrease of a liver function, and local inflammation, is increased.

There is a hypothesis that hair loss or baldness in adult men is caused due to the excessive male hormone called dihydrotestosterone (DHT or 5α-dihydrotestosterone) (J. Urol. 179 (6): 2333-2338). The normal range of DHT is 2.02 to 12.38 ng/ml for males and 0.87 to 5.99 ng/ml for females. However, persons, who are generally in the process of hair loss, tend to show significantly higher DHT values than that of a normal person, and attention is strongly paid to an opinion that hair follicles shrink and hair loss occurs due to an increase in DHT is strongly appealed.

Accordingly, most compositions for preventing hair loss or promoting hair regrowth limit the DHT, which is a male hormone, in order to promote hair regrowth. Accordingly, the sexual function of the male is degraded so that males may hesitate use the compositions.

### SUMMARY OF THE INVENTION

An object of the present invention is to provide a composition for preventing hair loss of modern people resulting from various causes, producing hair follicles, and promoting hair regrowth without side effects. In more detail, an object of the present invention is to provide a composition including effective ingredients of edible crickets for promoting hair regrowth, preventing hair loss, and improving hair follicles and provided in the form of one selected from the group including formulations of capsules, powders, granules, tablets, pills, and drink.

The United Nations has designated insects as future food sources (Edible insects Future prospects for food and feed security: FAO) as well known to those skilled in the art. This is because the insects have vigorous reproductive abilities to increase the number of individuals within a short period, and abundant nutrients including proteins. In addition, among the insects, the fact that crickets improve liver functions and relieve hangovers is already known through the research results of the Agricultural Development Agency.

The inventor of the present invention can recognize that edible crickets help persons who take in edible crickets to improve live functions and relieve hangover after drinking. In addition, in the process in which the persons take in the edible crickets to improve the live functions, the inventor of the present invention has found out a surprising fact by chance. The fact is that the hairs of the persons become rich with the improved live function as the persons take in the edible crickets. The found fact provides a clue to remarkably solve problems related to the hair loss and the hair regrowth, which are not solved in the related arts. The inventor of the present invention carries out many experiments in order to obtain objective test results for determining the probability of solving the problems, and the above effectiveness of the edible crickets can be confirmed.

According to the present invention, effective ingredients of edible crickets can be continuously supplied to the human body to prevent hair loss, promote hair growth, and improve hair follicles.

In detail, the edible cricket powders were taken in a dose of 30 g 2-3 times on a daily basis while maintaining a maximum daily average dose of 100 g. The edible cricket powders are supplied to a human body for a long term of 2-5 months or more to produce effects of preventing hair loss, improving hair follicles, and promoting hair regrowth.

The useful ingredients of the edible crickets may be obtained not only in the form of edible cricket powders, but in the form of extracts obtained in a typical extraction manner, and then may be processed in the form of various formulations to be supplied.

It is desirable to use an extraction manner that minimizes the destruction of nutritional ingredients of crickets. In particular, the vacuum low-temperature extraction manner has an advantage in that the boiling point is lowered in a vacuum state and water boils even at a low temperature (40 to 90 degrees Celsius) so that the extracts can be obtained from the crickets without the destruction of ingredients of substances sensitive to heat or substances reacting with air.

Hereinafter, details of a method of preparing a composition including cricket powders according to one embodiment will be described below. According to the present invention, a method of preparing a composition for an edible hair growth agent using formulated ingredients of edible crickets includes
a) cooling an edible cricket by washing the edible cricket,
b) making cricket powders by drying and pulverizing the edible crickets prepared in step a),
c) making a solid matter by drying and compressing the edible crickets prepared in the step a), and
d) mixing the formulation prepared in steps b) and c) with an oral excipient to prepare an oral preparation.

According to the present invention, a subject, who has undergone hair loss for 10 years or more, takes edible crickets, which are dried, pulverized, and formulated, in a one-time dose of 30 g 2 times on a daily basis while maintaining a maximum daily average dose of 100 g. The subject taking the formulation is photographed on 30^{th} date, 50^{th} date, a 70^{th} date, and a 90^{th} date, so that the hair regrowth state of the subject is observed and test results are exactly provided.

As described above, according to the present invention, when powders or extracts of the edible crickets are used as hair growth agents, the edible crickets can produce effects of promoting hair regrowth, preventing hair loss, and improving hair follicles. Therefore, although the mechanism that the edible cricket ingredients promote hair regrowth, prevent hair loss, and improve hair follicles is not exactly known, it is estimated that abundant nutritional ingredients contained in the edible crickets interact with each other to produce the above effects.

In addition, according to the present invention, the hair loss can be effectively prevented, the hair follicles can be improved, and the hair regrowth effect can be obtained without the degradation of the sexual function of a male. This is because ingredients such as iron (Fe), zinc (Ze), and copper (Cu) contained in the edible crickets inhibit DHT, which is a male hormone, pointed as the cause of the hair loss. In addition, arginine, which is a kind of a protein, helps penis blood vessel enlargement to complement the degradation of the sexual function resulting from iron (Fe), zinc (Ze), and copper (Cu) and to minimize the side effects in the degradation of the sexual function, or to increase the sexual function.

Since the above case has never been reported worldwide until now, the added value may be increased if a product using the edible crickets is commercialized.

### BRIEF DESCRIPTION OF THE DRAWINGS

FIG. 1 shows a process for producing an oral hair regrowth product composition including edible crickets.

FIG. 2 shows photographs taken at 30 days, 50 days, 70 days, and 90 days during the hair regrowth experiment of a 45-year-old male who has been continuously suffering hair loss for the past 10 years.

FIGS. 3 to 6 are photographs showing the result of the hair regrowth progress test by the hair regrowth experiment.

FIG. 7 is a photograph showing the hair regrowth state of an experimental group fed with cricket feeds and a control group fed with normal feeds.

### DETAILED DESCRIPTION OF THE INVENTION

Since the edible cricket formulations less produce side effects, the formulations are safe. In addition, since the formulations have higher absorption rates than those of synthetic medicines or supplements to promote hair regrowth and prevent hair loss, the formulations are used for medicines, supplements and cosmetics. Particularly, since the edible cricket formulations contain iron (Fe), zinc (Ze), copper (Cu), and biotin (Biotin, Vitamin B7) which are higher than the nutritional ingredients contained in vitamins sold on the market and contain a large amount of amino acids constituting hair, the edible cricket formulations produce excellent effects.

In addition, as shown in an experimental result, since big effects can be easily produced within a shorter period of time.

The present invention relates to a composition including an effective ingredient of edible crickets to promote hair regrowth, prevent hair loss, and improve hair follicles. Hereinafter, the composition including the effective ingredient of the edible crickets according to the present invention will be described with reference to accompanying drawings.

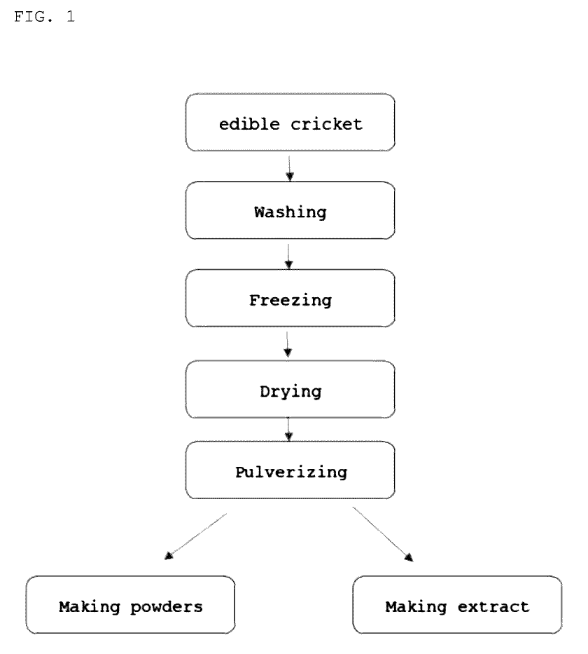

Healthy edible crickets are washed, frozen at a subzero temperature, and dried, and made in the form of powders. The above processes are shown in FIG. 1.

After mixing 30 g of powders, which are obtained through the above processes, and water at the ratio of 1:1, the mixture is steamed and then mixed with 250 g of starch to be prepared in the form of a pill through a typical pill manufacturing manner.

In addition, 30 g of cricket powders, which are obtained above, or cricket extracts may be mixed with 50 g of citric acid, 5 g of oligosaccharide, 1 g of taurine, and 914 ml of purified water through a typical drink preparing manner, and the mixture may be sterilized and sealed to make a drink.

As described above, the prepared powders or the prepared extracts may be prepared in the form of a formulation of capsules, powders, granules, tablets, pills, or drinks according to the typical preparation manner for the oral medication.

The extraction methods, which are generally known, include distillation extraction methods, or vacuum low temperature extraction methods. Preferably, the vacuum low temperature extraction method can effectively extract useful ingredients from crickets without the destruction thereof. In addition, extracts may be obtained from the cricket powders through a generally-well known extraction method.

In addition, after the extracts are obtained from the crickets, the extracts may be included in one selected from the group of shampoo, hair rinse, hair tonic, hair gel, hair lotion, hair pack, hair spray, hair mousse, hair treatment, and hair dye.

### Experimental Example 1

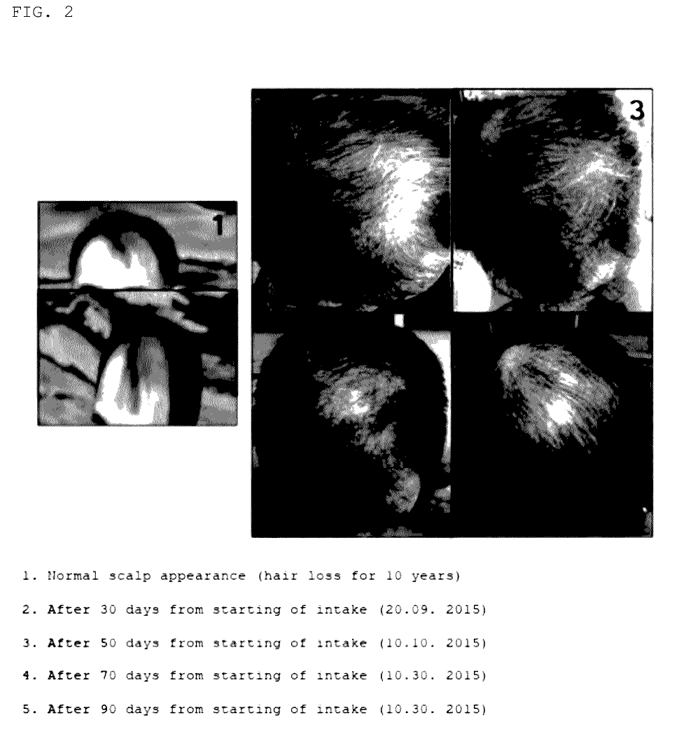

FIG. 2 shows a photograph of a 45-year-old male who has undergone continuous hair loss for the past 10 years. When considering results of studies on hair loss prevention and hair regrowth products, most studies are performed on hair regrowth when hair loss is less progressed in the early stage of the hair loss. According to the result of the present study, it can be easily recognized through the observation of photographs that hairs are produced, thickened, and grown within the shortest period of time (experimental periods: 30 days, 50 days, 70 days, and 90 days) with respect to hair-loss patients who chronically and continuously become bald for 10 years or more. It can be recognized through the present study that nutritional ingredients of edible crickets can produce significantly excellent hair regrowth effects.

As shown in photograph 1 of FIG. 2, hairs rarely are present on front and side portions of a head due to the hair loss continued for 10 years. However, as shown in photographs 2 to 5 of FIG. 2, it can be recognized through the present experiment that the hair regrowth effect of dried edible crickets is excellently produced after 30 days for which a user uniformly takes in a daily dose of dried edible crickets. In addition, it can be clearly recognized that hairs gradually become thicker throughout the head of the user as 30 days, 50 days, 70 days, and 90 days elapse after the experiment is started.

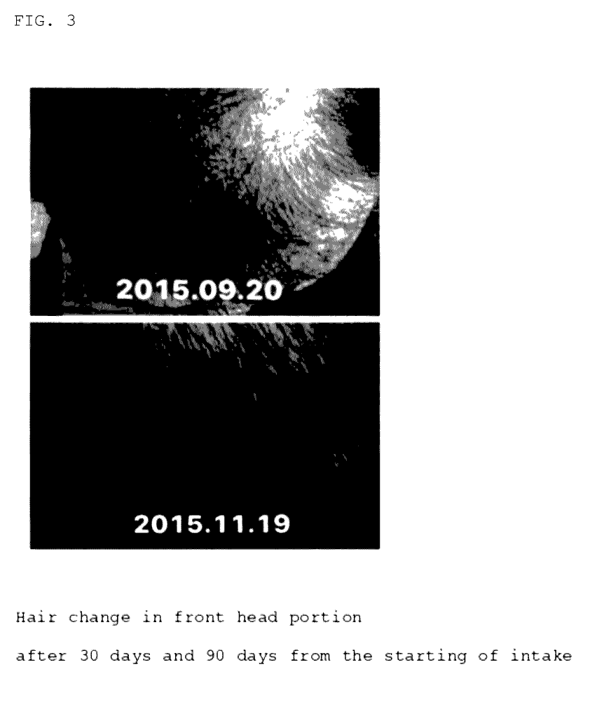

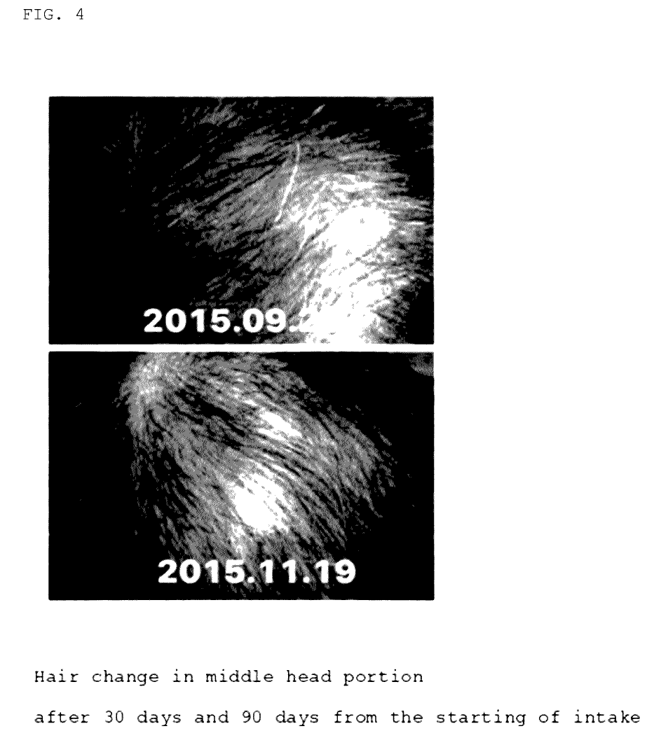

FIG. 3 illustrates photographs of front, middle, and rear parts of a head after 30 days or 90 days elapses from the experiment. As shown in FIG. 3, a larger amount of hairs are produced even after 30 days elapses from the experiment when comparing with an amount of hairs before the experiment is performed. Further, it can be recognized that significantly rapid hair regrowth effects are produced on the front part of the head after 90 days elapses from the experiment. In addition, it can be recognized through FIG. 4 that the whole hairs deviate from a hair loss stage of exhibiting a powerless hair state, gradually become thicker, and exhibit strong hair density.

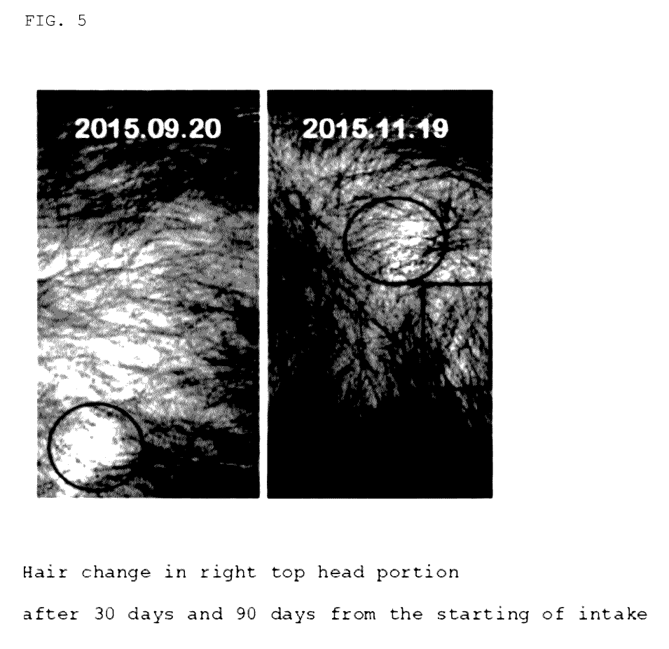

FIG. 5 illustrates cross-sectional increment comparison for a right top part of the head for more precise comparison between hair states shown in FIGS. 3 and 4. When comparing with the hair appearance after 30 days, the hair appearance after 90 days makes a great difference in hair density, and shows a thicker hair shaft as clearly shown at a glance.

Further, FIG. 5 shows important information on hair regrowth. In other words, a patient having chronically undergone hair loss for 10 years shows a distinct difference in hair regrowth only in 90 days. In a left part of FIG. 5, which shows the hair appearance after 30 days, black dots in a circle represent hairs which are newly grown. In addition, it can be recognized from a right part of FIG. 5 showing the hair appearance after 90 days that hairs in the circle become rich. In addition, as shown in the photograph of the hair appearance after 90 days, black dots appear between hairs in circle and rectangular parts, which represent that hair shafts starts growing out of a scalp dermis.

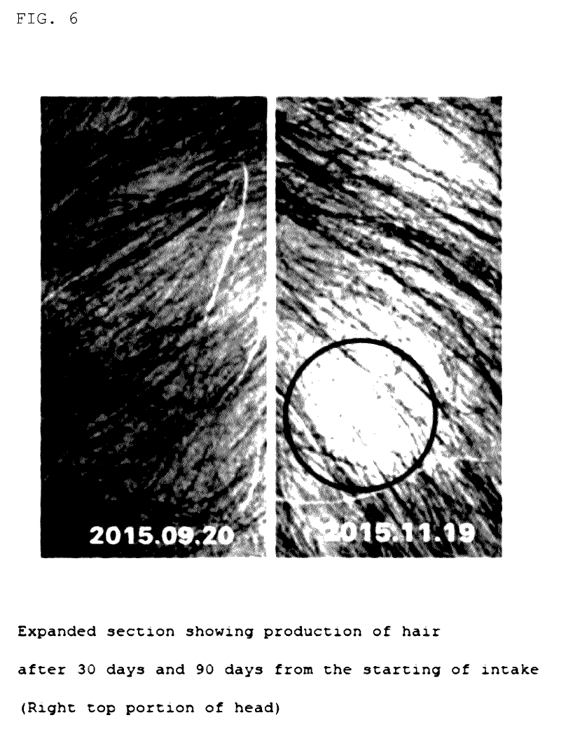

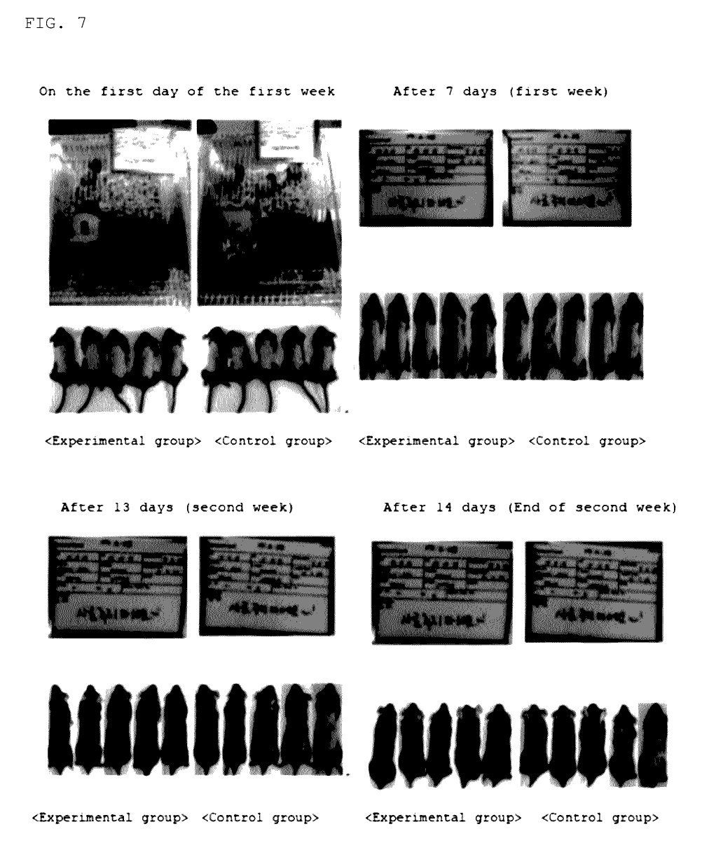

FIG. 5 shows that hairs are richly re-grown from the part of the new hair shafts, which are formed after 30 days, after 90 days in the left part of FIG. 5, FIG. 6 is a photograph showing an expanded section of a top head part, which provides more important information. It can be recognized that new hair shafts formed after 30 days from the experiment, are grown to hairs after 60 days since then, that is, after 90 days from the experiment. As shown in a circle part of FIG. 6, new black dots, which are not absent in the right photograph of FIG. 5, are formed on the scalp of a user after 60 days. This shows a significantly important hair regrowth progress that hair regrowth is performed to form the hair shaft only in 60 days.

The results of the present study show that hair loss can be remarkably prevented and hair regrowth can be significantly improved by nutritional ingredients contained in the edible crickets as a patient chronically undergone hair loss for 10 years or more takes about 30 g of dried cricket powders or extracts twice a day.

### Experimental Example 2

Person A who has severely undergone hair loss is supplied with 30 g of edible cricket powders the same as that of the experimental example two times on a daily basis. Thereafter, the hair state of the head top part of the person A, which shows severely hair loss, is measured in the unit of one month.

TABLE 1

| | Head top part | | Head rear part | |
|---|---|---|---|---|
| | | Existing | | Existing |
| | | average | | average |
| | | hair | Density | hair |
| | Density (hairs/ | thickness | (hairs/ | thickness |
| | cm²) | (mm) | cm²) | (mm) |
| Initial stage | 65 | 0.029 | 86 | 0.038 |
| After 30 days | 63 | 0.031 | 82 | 0.040 |
| After 60 days | 81 | 0.035 | 98 | 0.043 |
| After 90 days | 93 | 0.042 | 115 | 0.052 |
| After 120 days | 116 | 0.050 | 132 | 0.068 |

### Experimental Example 3

Person B who has severely undergone hair loss is supplied with 30 g of edible cricket powders the same as that of the experimental example twice a day. Thereafter, the hair state of the head top part of the person B, which shows severely hair loss, is measured in the unit of one month.

TABLE 2

| | Head top part | | Head rear part | |
|---|---|---|---|---|
| | | Existing | | Existing |
| | | average | | average |
| | Density | hair | | hair |
| | (hairs/ | thickness | Density | thickness |
| | cm²) | (mm) | (hairs/cm²) | (mm) |
| Initial stage | 85 | 0.031 | 90 | 0.042 |
| After 30 days | 83 | 0.036 | 89 | 0.049 |
| After 60 days | 90 | 0.040 | 115 | 0.056 |
| After 90 days | 112 | 0.046 | 134 | 0.062 |
| After 120 days | 132 | 0.053 | 157 | 0.070 |

Clinical experiments in Experimental examples 2 and 3 are to measure the hair states of persons A and B close to partial baldness after edible cricket powders are continuously supplied to the persons A and B. According to the experimental examples 2 and 3, the number of hairs is continuously increased since 30^{th} date from the experiment except that the number of hairs is reduced in partial hair loss process until the 30^{th} date, so that the hair density is slightly reduced. In other words, new hair follicles are produced, so that the hair density is increased. Since the hairs in the newly produced hair follicles are thin, the average thickness of whole hairs is less. However, when only existing hairs are measured, the measurement result shows that the hairs are gradually thickened. According to the results of the experiment for 40 months, hair thicknesses in all of the head top part and the head rear part approximate hair thicknesses (0.075 mm) of a normal person.

### Experimental Example 4 Animal Experiment

The present experiment is commissioned to Inha University Hospital in order to test an influence exerted on the hair regrowth of C57BL/6 Mouse as edible cricket powders are fed to the C57BL/6 Mouse. This experiment is to test the influence exerted on the hair regrowth by rich proteins, unsaturated fatty acids, and minerals such as copper (C), iron (Fe), and zinc (Zn) contained in crickets.

The test is performed by depilating backs of 6-week-old male mice, making two groups, each group including five rates, and separately observing the two groups. Cricket compound feeds are fed into an experimental group, and standard solid compound feeds, which are radiation irradiated solid feeds for rodents (PMI LabDiet USA Oval pellet, Irradiation), are fed into a control group for distinguish between the experiments. For an experiment environment, light is turned on for 12 hours, and the rates freely take water sterilized after reverse osmosis treatment.

Hair regrowth is observed throughout 1, 2, 3, and 4 weeks by photographing the depilated backs of the mice belonging in each group. In the first week, the mice in the groups show 0-19% of hair growth, which does not show the difference between the groups. In the second weeks, all five mice belonging to the experimental group show 80-100% of hair growth while the hair growth of the control group is shown by 20-39% from two mice, 40-59% from one mouse, 60-79% from one mouse, and 80-100% of hair growth is shown from one mouse ((P<0.05), so that meaningful results are obtained. In third and fourth weeks, all five mice in both groups show 80-100% of hair growth, which does not make the difference between both groups. It is estimated that the hair regrowth is achieved in an early stage since the edible crickets have rich nutritional ingredients as compared to those of existing feeds.

<Detailed Analysis of Hair Regrowth in Second Week>

<Experimental Results of Mouse>

| Hair regrowth | | |
|---|---|---|
| effect | Control group | Experimental group |
| 1 (10-19% hair | 0 | 0 |
| regrowth) | | |
| 2 (20-39% hair | 2 | 0 |
| regrowth) | | |
| 3 (40-59% hair | 1 | 0 |
| regrowth) | | |
| 4 (60-79% hair | 1 | 0 |
| regrowth) | | |
| 5 (80-100% hair | 1 | 5 |
| regrowth) | | |
| Average hair | 3 | 5 |
| regrowth value | | |

TABLE 4
Test Statistic b

| | effect |
|---|---|
| U of Mann-Whitney | 2.500 |
| W of Wilcoxon | 17.500 |
| Z | −2.362 |
| Approximate | .018 |
| significance probability | |
| (both sides) | |
| Exact significance | .032 |
| probability [2 * (shortened | |
| significance probability)] | |

As a result of measuring the change in the body weight of the mice after feeding the mice for four weeks, the body weight of the mice in the control group is increased by 2.36 kg on average, while the body weight of the mice in the experimental groups is increased by 2.46 kg on average.

Although the experiments are performed using edible twin crickets according to the present invention, it is predictable to those skilled in the art that experiments with other cricket species produce the same or similar effects.

Although the results of the experiments with edible cricket powders are obtained as described above, it is obvious to those skilled in the art that edible cricket extracts produce the same or similar effects. Preferably, after the extracts are obtained from the edible cricket powders for the easy intake or application, the user may take in the extracts and other ingredients or apply the extracts and other ingredients to the scalp of the user, so that the same or similar effects may be produced.

<Influences Exerted on Hair Loss, Hair Regrowth, and Hair Follicles by Effective Ingredients of Edible Crickets>

Hereinafter, description will be made regarding several nutritional factors contained in the edible crickets and significantly affecting hair follicles causing hair loss. In general, the nutritional ingredients related to hair regrowth are mainly classified into minerals, vitamins, and amino acids. Minerals affecting hair growth include iron (Fe), zinc (Zn), and copper (Cu). Vitamins affecting the hair growth include biotin (vitamin B7), and amino acids affecting the hair growth include keratin, glutamic acids, methionine, arginine, and cysteine. In addition, blood circulation affects the hair regrowth. Since all nutritional ingredients are contained in the edible crickets, it is estimated that the edible crickets have excellent functions for hair growth effects.

In addition to the nutritional ingredients that affect hair growth, the edible crickets contain 20 amino acids including essential amino acids that make up proteins, and contain a large amount of omega-3 fatty acids, vitamins, and minerals, which are known through study analysis (see Rumpold, B A; Schluter, O K. & Quot; Nutritional Composition and Safety Aspects of Edible Insects & quot; Molecular Nutrition & Food Research; May, 2013; 57; 5; Quot; P802-823 & quot).

According to the present invention, it can be estimated that the effective ingredient of the edible cricket containing such a large amount of ingredient is continuously administered and the nutrition is supplied to the scalp, thereby positively affecting hair growth and hair loss prevention

For reference, the nutritional ingredients of the edible crickets are shown from following table.

TABLE 5
Contents of minerals in edible crickets (mg/100 g)

| | | Growing | |
|---|---|---|---|
| | Imago | insect | Larva |
| Calcium | 132.14-210 | 1290 | 120.09 |
| Potassium | 1126.62 | | 1537.12 |
| Magnesium | 80-109.42 | 160 | 98.69 |
| Phosphorous | 780-987.79 | 790 | 110.44 |
| Sodium | 435.06 | | 589.52 |
| Iron | 6.27-11.23 | 19.68 | 9.26 |
| Zinc | 18.64-21.79 | 15.91 | 29.69 |
| Manganese | 2.97-3.73 | 5.28 | 3.89 |
| Copper | 0.85-2.01 | 0.96 | 2.23 |
| Selenium | 0.06 | | 0.04 |

In other words, although it is well known to those skilled in the art that the nutritional ingredients are contained in the edible crickets as shown in table 1, it is never known to those skilled in the art that the edible crickets containing the nutritional ingredients are useful for preventing hair loss, improving hair follicles, and promoting hair regrowth.

Such an edible cricket ingredient ratio produces an excellent effect on hair growth. Even if edible crickets are not used, a composition having the edible cricket ingredient ratio may have a hair growth effect. Specifically, a composition for preventing hair loss and promoting hair growth includes 6.79-11.23 mg of Fe, 18.64-21.79 mg of Ze, 0.8-2.01 mg of Cu, 57.3-61 mg arginine, 104.4-104.9 mg of glutamic acids, 14.6-19.6 mg of methionine, and 8.3-9.8 mg of cysteine.

TABLE 6
Comparison table of ingredients according to foods
(mg/100 g)

| | | | | Argi- | Glutamic | | |
|---|---|---|---|---|---|---|---|
| | Fe | Zn | Cu | nine | acid | Ethionine | Cysteine |
| Kidney | 8 | 2.5 | 0.75 | 620 | 2013 | 284 | 124 |
| bean | | | | | | | |
| Milk | 0.1 | 0.4 | — | 199 | 945 | 161 | 4 |
| Chicken | 1 | 1.4 | 0.09 | 425 | 562 | 193 | 51 |
| Beef | 0.3 | 5.7 | 0.08 | 380 | 812 | 130 | 3 |
| Cricket | 11.23 | 21.79 | 2.01 | 5520 | 10490 | 1960 | 980 |

(1) Iron (Fe)

Our body is made up of fat, protein, and many minerals. When the above ingredients are insufficient or deficient, blood may not be produced or unhealthy blood may be produced. Iron carries oxygen to tissues from the center of the hemoglobin in red blood cells. However, for hair growth, the supplying of nutrients to hair follicles, that is, blood circulation becomes very important. The iron seems to effectively transfer oxygen to the hair follicles and scalp to help hair grow.

(2) Zinc (Ze)

Zinc intake stimulates the metabolism of cells and tissues in the body, helps the collagen synthesis with Vitamin C, and prevents hair aging. In addition, zinc is a significantly important mineral to play various roles, such as diabetes prevention which maintains blood glucose by promoting insulin secretion by a pancreas, immunity improvement, stamina strengthening, kidney filtration, prevention of high blood pressure, prevention of shoulder pain and stiffness, the prevention of osteoporosis, bone density improvement, anemia prevention as a source material of a red blood cell, and anti-oxidation to remove oxygen radicals. Zinc is a component that inhibits male hormone (DHT) that adversely affects hair loss and contained in edible crickets in large amounts.

(3) Copper (Cu)

Copper (Cu), which is a component of enzymes and proteins in the body, is contained in the body by about 100-150 mg in the case of adults. Cu is most abundant in the liver and brain and exists in the order of a heart, a kidney, a pancreas, a spleen, a lung, a bone, and a muscle. Cu makes a red blood cell of blood together with iron and increases the absorption and utilization of iron to maintain a cardiovascular system. In addition, Cu participates in the formation of collagen and elastin to maintain bone, cartilage, and skin tissue and to improve an immunity function. Further, Cu is an essential nutritional ingredient for inflammatory diseases such as arthritis, spondylitis and an ulcer.

However, Cu, which is a substance for inhibiting 5-alpha-reductase from causing hair loss, enlarges hair follicles, increases hair density, and prevents hair loss by an anti-inflammatory action.

(4) Biotin (Vitamin B7)

Biotin is known as Vitamin H or Vitamin B7 to prevent hair loss, recover brittle nails, and make skin, hairs and nails healthy. Similarly other vitamins belonging to a vitamin B group, biotin is soluble in water but is not dissolved in an adipose tissue. Although biotin cannot be created in the human body, ‘good bacteria’ that are symbiotic to human cells synthesize biotin. Biotin is needed not only for good hair growth but also for digestion of food and the management of blood glucose. In addition, biotin is known as a substance to control cholesterol and blood glucose by helping to decompose carbohydrates, fats and proteins.

Therefore, the main symptoms of biotin deficiency are hair loss, hyperlipidemia, hyperglycemia, hepatomegaly, depression, cutaneous sense malfunction, myalgia muscle pain, and fatigue accumulation.

Crickets are rich repositories of vitamins such as vitamin A, vitamin E, vitamin C, vitamin B1, vitamin B2, vitamin B3, vitamin B5, vitamin B7, and vitamin B9 that are necessary for the health of a human body. Especially, crickets contains vitamin B7, which is useful for preventing hair loss, for promoting hair regrowth, and for skin, by 55.19 mcg/100 g.

(5) Protein Amino Acids that Affect Hair Loss

Keratin, which forms hairs and is called stratum corneum, includes 18 protein amino acids. Among them, there are proteins important to prevent hair loss and to promote hair regrowth by supplying nutrition to hair follicle and improving blood circulation flow, and the most representative amino acids constituting the proteins are arginine, glutamic acid, cysteine, and methionine as well known in a medical field. Since all of the useful substances are contained in the edible crickets used for the study according to the present invention, the edible crickets have the above effects.

Since all amino acids for hair regrowth are sufficiently contained in the edible crickets, if a user constantly takes in the edible crickets as a meal, the hair regrowth is achieved within a short period of about three months, hairs are thickened, and new hairs are produced as easily observed in the enlarged photograph (see FIGS. 2-6).

\textcircled{1} Arginine

The arginine amino acid, which is one of the three major components of keratin, is a protein component that reacts with the endothelial cells of a blood vessel wall to activate nitric oxide production, thereby improving blood flow of hair follicle cells to directly cause hair loss to promote hair growth. The edible crickets contain a larger amount of arginine in the range of 57.3-61 mg/100 g.

\textcircled{2} Glutamic Acids

As nourishment is supplied to hairs, a hair root absorbs nourishment for division and proliferation to grow a new hair cell. Most hair components include scleroprotein, that is, keratin. Amino acids constituting keratin in hair include mainly cysteine, which are followed by glutamic acid. The dry crickets contain a large amount of glutamic acid in the range of 104.4-104.9 mg/100 g.

\textcircled{3} Methionine and Cysteine Containing Sulfur

Methionine and cysteine are amino acids containing sulfur components that exert a significant influence on hair follicles and is involved in blood circulation. Methionine and cysteine containing the sulfur component are essential minerals for keratinization in hair, skin, nails and toenails, and cysteine occupies the most part of keratin, that is, 14-18% of keratin components as known in the art. The two amino acids containing the sulfur components are known to improve the production of collagen tissues essential for helping blood circulation to eliminate dandruffs from scalp so that the hair regrowth is promoted. The dried crickets contain larger amounts of methionine and cysteine, and the content of the methionine content is in the range of 14.6-19.6 mg/100 g and the content of the cysteine is in the range of 8.3-9.8 mg/100 g.

As described above, the edible crickets contain abundant minerals, vitamins, and protein amino acids to help the prevention of the hair loss and the promotion of the hair regrowth. Accordingly, significantly remarkable results are obtained within three months of the hair regrowth experiment. Edible crickets abundantly contain Zn, Fe, Cu, vitamin A, and biotin (vitamin B7), and contains large amounts of methionine and cysteine containing sulfur, glutamic acids, and arginine amino acids including nitrogen oxygen in large amount, so that the hair regrowth can be remarkably promoted.

The contents of main nutritional ingredients contained in the edible crickets may provide an idea of the desirable contents of nutritional ingredients necessary for the promotion of hair regrowth. Accordingly, there may be provided a composition for preventing hair loss and promoting hair growth includes 6.79-11.23 mg of Fe, 18.64-21.79 mg of Ze, 0.8-2.01 mg of Cu, 57.3-61 mg arginine, 104.4-104.9 mg of glutamic acids, 14.6-19.6 mg of methionine, and 8.3-9.8 mg of cysteine.

However, all nutritional ingredients, which are excessively taken in, may cause side effects as known as a common sense. Accordingly, although the edible crickets contain ingredients useful for the hair regrowth, the edible crickets do not always produce excellent effects on the prevention of hair loss, the improvement of hair follicle, and the promotion of hair regrowth. Nonetheless, it can be confirmed that the interaction of the nutritional ingredients contained in the edible crickets produces excellent effects that hair regrowth is rapidly accelerated, hair thickness is increased, and gray hairs are turned into black hairs. Therefore, although the interaction among the nutritional ingredients contained in crickets and the mechanism of the interaction are not known, the nutritional ingredients have the remarkable effects on the prevention of the hair loss, the promotion of the hair regrowth, and a male sexual function without side effects.

What is claimed is:

1. A composition for preventing hair loss, improving a hair follicle, or promoting hair regrowth, the composition comprising an edible cricket powder or an edible cricket extract.

2. The composition of claim 1, wherein the composition is included in one selected from a group including shampoo, hair rinse, hair tonic, hair gel, hair lotion, hair pack, hair spray, hair mousse, hair treatment, and hair dye.

3. A pharmaceutical composition for promoting hair regrowth, comprising the composition according to claim 1.

4. A pharmaceutical composition for preventing hair loss, comprising the composition according to claim 1.

5. A pharmaceutical composition for improving a hair follicle, comprising the composition according to claim 1.
